(19) United States
(12) Plant Patent Application Publication
(10) Pub. No.: US 2003/0177557 P1
(43) Pub. Date: Sep. 18, 2003

(54) Alstroemeria plant named 'Tara'

(75) Inventor: Robert Adrian Goemans, Lagness (GB)

Correspondence Address: FOLEY AND LARDNER, SUITE 500, 3000 K STREET NW, WASHINGTON, DC, 20007, US

(73) Assignee: Wulfinghoff Alstroemeria B.V.

(21) Appl. No.: 10/097,252

(22) Filed: Mar. 15, 2002

(51) Int. Cl. A01H 5/00

(52) U.S. Cl. PLT/309

(57) ABSTRACT

A new and distinct Alstroemeria plant named ‘Tara’ characterized by growth height of 10-30 cm under greenhouse conditions; suitable for any pot size over 7 cm.

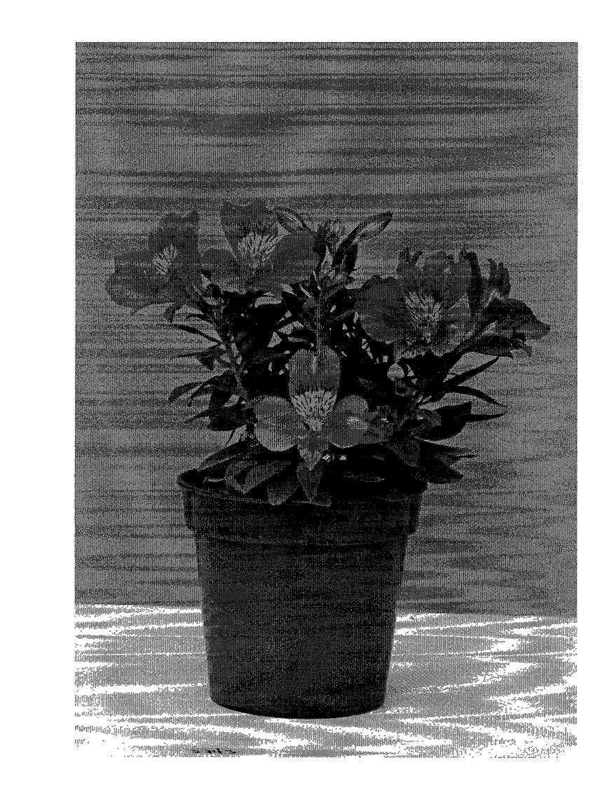

### LATIN NAME OF THE GENUS AND SPECIES OF THE PLANT CLAIMED

Genus: Alstroemeria; Species unknown

### VARIETY DENOMINATION

‘Tara’

### BACKGROUND OF THE INVENTION

The present invention comprises a new and distinct cultivar of Alstroemeria plant, hereinafter referred to by the cultivar name ‘Tara’.

‘Tara’ originated from a hybridization made in a controlled breeding program in Chichester in Sussex, United Kingdom by the inventor Robert Adrian Goemans. The female parent is an unknown cultivar of unknown origin (unpatented). The male parent of ‘Tara’ is a selection of the cultivar ‘Butterfly’.

‘Tara’ was discovered and selected as one flowering plant within the progeny of the stated parentage by Robert Adrian Goemans in 1997 in a controlled environment in Chichester, United Kingdom. The first act of asexual reproduction of ‘Tara’ was accomplished when vegetative cuttings were taken from the initial selection in May, 1999 in a controlled environment in Chichester, United Kingdom. Horticultural examination of selected units initiated in 1999 has demonstrated that the combination of characteristics as herein disclosed for ‘Tara’ are firmly fixed and retained through successive generations of asexual reproduction.

‘Tara’ has not been observed under all possible environmental conditions. The phenotype may vary significantly with variations in environment such as temperature, light intensity and day length, without any change in genotype. The following observations, measurements and comparisons describe plants grown in Chichester, Sussex, United Kingdom under greenhouse conditions which approximate those generally used in commercial practice.

### BRIEF SUMMARY OF THE INVENTION

The following traits have been repeatedly observed and are determined to be basic characteristics of which in combination distinguish this Alstroemeria as a new and distinct cultivar:

1. Growth height 10-30 cm under greenhouse conditions; suitable for any pot size over 7 cm.

There are no commercial cultivars known to the present inventor, similar in comparison to ‘Tara’.

### BRIEF DESCRIPTION OF THE DRAWINGS

The accompanying photographic drawing shows a typical specimen of the new variety with colors as accurate as possible.

The photographic drawing shows the flowers and foliage of ‘Tara’.

### DETAILED BOTANICAL DESCRIPTION

In the following description, color references are made to The Royal Horticultural Society Colour Chart (RHS). The color values were determined in the afternoon of Sep. 16, 2000 under normal daylight conditions in Wateringen, The Netherlands.

Classification:

Botanical.—Alstroemeria.

Commercial.—Alstroemeria cv. Tara.

Plant:

Form.—Pot Plant.

Height.—10-30 cm (Dutch greenhouse conditions).

Growth.—Bushy-dwarf growth habit.

Foliage.—Quantity: 10-20 leaves on each centimeter of stem, depending on growth stage Size of leaf: Length: 70-90 mm; width: 30-35 mm Shape of leaf: Oblong-ovate Color: Upper side is dark green, RHS 137 A to RHS 137 B underside is light-green, RHS 137 C.

Rhizomes.—Color: White, RHS 158 A to RHS 158 D Size: Length 10-50 mm; diameter 6-15 mm, depending on age.

### INFLORESCENCE

Bud:

Form.—Tubular pointed.

Diameter.—10-20 mm.

Length.—15-25 mm.

Peduncle.—20-25 mm, medium-green RHS 145 A.

Color.—Red, RHS 46A to RHS 46 B.

Flower.—Size: Medium, length: 40-60 mm; width: 40-60 mm Borne: Medium number of branches in umbel Shape: Broad obovate

Color.—Upper surface: Red, RHS 47 A to RHS 47 D Under surface: Yellow at base, RHS 13 A to RHS 13 D Markings: Black stripes on all petals, RHS 202A.

Peduncle.—Length: 40-50 mm Color: Medium-green, RHS 145 A.

Lasting quality.—4-6 weeks.

Reproductive Organs:

Stamens.—6.

Anthers.—Yellow RHS 13 A.

Filaments.—Red RHS 50 A to RHS 50 B.

Pistils.—1, color red-pink, RHS 50 B to RHS 50 C.

Stigma.—Color pink-white, RHS 56 A to RHS 56 C.

1. I claim a new and distinct cultivar of Alstroemeria plant named ‘Tara’, as described and illustrated herein.
